(19) United States
(12) Patent Application Publication
(10) Pub. No.: US 2025/0121071 A1
(43) Pub. Date: Apr. 17, 2025

(54) REAGENTS AND METHODS FOR PREPARING DERIVATIZED RNA MOLECULES AND METHODS OF USE THEREOF

(71) Applicants: The Broad Institute, Inc., Cambridge, MA (US); MASSACHUSETTS INSTITUTE OF TECHNOLOGY, Cambridge, MA (US)

(72) Inventors: Xiao Wang, Cambridge, MA (US); Abhishek Aditham, Cambridge, MA (US); Franklin Kostas, Cambridge, MA (US)

(21) Appl. No.: 18/932,299

(22) Filed: Oct. 30, 2024

Related U.S. Application Data
(63) Continuation of application No. PCT/US2023/067254, filed on May 19, 2023.
(60) Provisional application No. 63/343,830, filed on May 19, 2022.

(51) Int. Cl. A61K 47/54; C12N 9/00; C12P 19/38

(52) U.S. Cl. CPC A61K 47/549; C12N 9/93; C12P 19/38; C12Y 605/01

(57) ABSTRACT

This invention provides compositions, reagents, methods, and kits for producing derivatized RNA molecules, particular mRNA molecules encoding a polypeptide and in particular a therapeutic protein, derivatized by linkage to a peptide, aptamer, synthetic DNA or RNA oligonucleotide or molecular probe, capable of targeting the derivatized RNA molecules to a particular subcellular location in a target cell.

### CROSS REFERENCE TO RELATED APPLICATIONS

This application claims priority to U.S. Provisional Application No. 63/343,830, filed May 19, 2022, and International Application No. PCT/US2023/067254, filed May 19, 2023, the contents of which are hereby incorporated by reference in their entirety.

### REFERENCE TO AN ELECTRONIC SEQUENCE LISTING

The instant application contains a Sequence Listing that has been submitted electronically and is hereby incorporated by reference in its entirety. The Sequence Listing was created on Oct. 30, 2024, is named “21-1400-US-CON_SequenceListing_ST26.xml,” and is 2,843 bytes in size.

### TECHNICAL FIELD

This invention relates to compositions, reagents, and methods for producing and delivering derivatized RNA molecules, particular mRNA molecules encoding a polypeptide and in particular a therapeutic protein, derivatized by linkage to a peptide, aptamer, synthetic DNA or RNA oligonucleotide or molecular probe, capable of targeting the derivatized RNA molecules to a particular subcellular location in a target cell.

### BACKGROUND

RNA therapeutics have recently developed rapidly as a field, as evidenced by recent clinical demonstrations of successful mRNA vaccines against SARS-COV-2. See, Polack et al., 2020, Safety and Efficacy of the BNT162b2 mRNA Covid-19 Vaccine. N. Engl. J. Med. 383, 2603-2615; Lombardi et al., 2021, Mini Review Immunological Consequences of Immunization With COVID-19 mRNA Vaccines: Preliminary Results. Front. Immunol. 12, 657711. mRNA's inherent programmability and relative ease of production underlie its potential to supplant conventional protein-based therapeutics. See, Sahin et al, 2014, mRNA-based therapeutics—developing a new class of drugs,” Nature reviews Drug discovery 13: 759-780. In addition to demonstrated clinical applications as exemplified by COVID vaccines, mRNA has been used experimentally to express vascular regeneration factors and to generate vaccines against influenza and Zika virus. See, Zangi et al., 2013, Modified mRNA directs the fate of heart progenitor cells and induces vascular regeneration after myocardial infarction, Nature biotechnology 31:898; Bahl et al., 2017, Preclinical and clinical demonstration of immunogenicity by mRNA vaccines against H10N8 and H7N9 influenza viruses, Molecular Therapy 25:1316-1327; Richner et al., 2017, Modified mRNA vaccines protect against Zika virus infection, Cell 168: 1114-1125.

Previous work has shown that incorporation of chemically modified nucleotides (i.e., pseudouridine and 5-methylcytidine) significantly reduces mRNA toxicity and increases protein expression, compared to mRNA consisting of only unmodified nucleotides. See, for example, Karikó et al., 2005, Suppression of RNA recognition by Toll-like receptors: the impact of nucleoside modification and the evolutionary origin of RNA, Immunity 23:165-175.

While current mRNA therapies have demonstrated robust expression and efficacy in niche in vivo applications (e.g. vaccines), current designs do not permit there to be adequate control over mRNA subcellular trafficking and distribution. Controlling the subcellular localization of mRNA translation is paramount for protein replacement therapies, as this permits direct targeting of products to their functional locations in a cell; see, Holt and Bullock, 2009, Subcellular mRNA localization in animal cells and why it matters, Science 326: 1212-1216; Das et al., 2021, Intracellular mRNA transport and localized translation, Nature Reviews Molecular Cell Biology 22:483-504. Such advances could enable more efficient production of functional membrane proteins, secreted products, and organelle-specific therapies.

Direct encoding of regulatory sequences into mRNA could potentially alter localization, as these could theoretically be recognized by proteins that traffic mRNAs to subcellular regions of interest. However, this strategy can face several limiting factors, including: 1) the use of modified nucleotides in therapeutic mRNAs, which are not necessarily recognized by endogenous protein binders; 2) low or absent expression of binding/trafficking proteins in target cells; or 3) the need to discover specific RNA-binding proteins targeting subcellular regions of interest. See, Holt & Bullock, 2009, id.; Das et al., 2021, id.

There thus remains a need in this art for reagents and methods for producing and using compositions comprising derivatized RNA molecules, in particular mRNA molecules encoding polypeptides such as therapeutic proteins, for specific targeting to subcellular locations in targeted cells to produce a useful, particularly a therapeutically useful, phenotypic effect on the recipient cells.

### SUMMARY OF INVENTION

This invention provides compositions, reagents, methods, and kits comprising derivatized RNA molecules preferably encoding a polypeptide, wherein the RNA molecule further comprises one or more of a peptide, aptamer, synthetic DNA or RNA oligonucleotide or molecular probe, capable of targeting the derivatized RNA molecules to a particular subcellular location in a target cell.

In certain embodiments, the derivatized RNA molecules comprise one or a plurality of chemically modified nucleotides, including but not limited to pseudouridine, N1-nethylpseudouridine, 5-methylcytidine, or a combination thereof. In embodiments intended for delivery to eukaryotic cells the derivatized RNA molecules comprising mRNA molecules each comprise a eukaryotic cap incorporated at the 5′ end of the derivatized RNA molecule. Further in embodiments intended for delivery to eukaryotic cells the derivatized RNA molecules each comprise a polyadenylate sequence (a poly A) incorporated at the 3′ end of the derivatized RNA molecule.

In certain embodiments the peptide, aptamer, synthetic DNA or RNA oligonucleotide or molecular probe, capable of targeting the derivatized RNA molecules to a particular subcellular location in a target cell is linked to the derivatized RNA molecule, in particular embodiments by a covalent linkage. In the structure of the derivatized RNA molecules provided by this invention the peptide, aptamer, synthetic DNA or RNA oligonucleotide or molecular probe, capable of targeting the derivatized RNA molecules to a particular subcellular location in a target cell is linked to the RNA in a manner that does not inhibit or reduce translation of mRNA embodiments thereof in the target cell. In certain other embodiments the peptide, aptamer, synthetic DNA or RNA oligonucleotide or molecular probe, capable of targeting the derivatized RNA molecules to a particular subcellular location in a target cell is linked to the RNA in a manner (e.g. chemical conjugation, enzymatic ligation, non-covalent tethering etc.) that enhances translation of mRNA, protein folding, post-translational modification of proteins, and subcellular protein trafficking (e.g. via ER localized translation, mitochondrial localized translation, cytoskeleton anchored translation, cytoplasmic membrane-anchored translation) embodiments thereof in the target cell. In particular embodiments peptide, aptamer, synthetic DNA or RNA oligonucleotide or molecular probe, capable of targeting the derivatized RNA molecules to a particular subcellular location in a target cell is linked to the RNA in the poly A tail or other portions of the mRNA, advantageously in untranslated regions thereof.

In certain embodiments the derivatized RNA molecules comprise an untranslated region (UTR) wherein the peptide, aptamer, synthetic DNA or RNA oligonucleotide or molecular probe, capable of targeting the derivatized RNA molecules to a particular subcellular location in a target cell is linked thereto. In certain embodiments the RNA molecule is an mRNA molecule that encodes a polypeptide. In certain embodiments the UTR is located at the 5′ end of the derivatized RNA molecule. In other embodiments the UTR is located at the 3′ end of the derivatized RNA molecule.

The invention provides embodiments of the derivatized RNA molecules that are targeted to the endoplasmic reticulum of the target cell. Also provided are embodiments of the derivatized RNA molecules that are targeted to the mitochondria of the target cell. Further provided are embodiments of the derivatized RNA molecules that are targeted to the target cell nucleus. In certain embodiments the derivatized RNA comprises a molecular probe capable of targeting the derivatized RNA molecules to a particular subcellular location in a target cell that binds to a target molecule at the targeted subcellular location. In particular embodiments, the peptide, aptamer, synthetic DNA or RNA oligonucleotide, or molecular probe targets the derivatized RNA molecule to a subcellular location in a recipient cell by recruiting chaperone proteins expressed in the recipient cell that shuttle the derivatized RNA molecule to the subcellular location. In certain particular embodiments the chaperone proteins are differentially expressed in the target cell. In other embodiments, the peptide, aptamer, synthetic DNA or RNA oligonucleotide, or molecular probe targets the derivatized RNA molecule to a subcellular location in a recipient cell by phase-mediated compartmentalization within the recipient cell.

In embodiments of the derivatized RNA provided by the invention comprising an mRNA encoding a polypeptide, in certain embodiments comprising a therapeutic protein. Polypeptides encoded by derivatized RNA molecules of the invention can include but are not limited to membrane-anchored proteins (including nuclear membrane, ER membrane, Golgi, cytoplasmic membrane), mitochondrial proteins, cytoskeleton proteins, C-tail anchored proteins, cellular junctions-anchored proteins (sarcomere in heart and muscle cells, gap junctions, neuronal synapses), secreted proteins, proteins with asymmetrical or heterogeneous subcellular distribution (e.g. proteins in embryonic cells, stem cells, epithelial cells, fibroblast, oligodendrocytes, immune cells etc.), and any other therapeutic proteins that need localized mRNA translation to maximize their production, folding, post-translational processing, trafficking, and the precision of subcellular locations.

The invention also provides methods for targeting derivatized RNA, particularly derivatized mRNA encoding a polypeptide, to a subcellular location in a recipient target, comprising the steps of introducing into a target cell a derivatized RNA, in particular said RNA that is an mRNA encoding a polypeptide, wherein the RNA is targeted to a subcellular location in the recipient target cell by the peptide, aptamer, synthetic DNA or RNA oligonucleotide, or molecular probe linked to the derivatized RNA molecule. In certain embodiments the derivatized RNA molecules comprise one or a plurality of chemically modified nucleotides, including but not limited to pseudouridine, 5-methylcytidine, or both pseudouridine and 5-methylcytidine. In embodiments intended for delivery to eukaryotic cells the derivatized RNA molecules each comprise a eukaryotic cap incorporated at the 5′ end of the derivatized RNA molecule. Further in embodiments intended for delivery to eukaryotic cells the derivatized RNA molecules each comprise a polyadenylate sequence (a poly A) incorporated at the 3′ end of the derivatized RNA molecule.

In certain embodiments of the methods provided herein, the derivatized RNA molecules comprise a peptide, aptamer, synthetic DNA or RNA oligonucleotide or molecular probe, capable of targeting the derivatized RNA molecules to a particular subcellular location in a target cell is linked to the derivatized RNA molecule, in particular wherein the linkage is a covalent linkage. In certain embodiments the peptide, aptamer, synthetic DNA or RNA oligonucleotide or molecular probe, capable of targeting the derivatized RNA molecules to a particular subcellular location in a target cell is linked to the RNA in a manner that does not inhibitor or reduce translation of mRNA embodiments thereof in the target cell. In certain alternative embodiments, the derivatized RNA molecules comprise a peptide, aptamer, synthetic DNA or RNA oligonucleotide or molecular probe, capable of targeting the derivatized RNA molecules to a particular subcellular location in a target cell is linked to the RNA in a manner that enhance translation of mRNA embodiments thereof in the target cell. In particular embodiments peptide, aptamer, synthetic DNA or RNA oligonucleotide or molecular probe, capable of targeting the derivatized RNA molecules to a particular subcellular location in a target cell is linked to the RNA in the poly A tail.

In certain embodiments methods provided herein, the derivatized RNA molecules comprise an untranslated region (UTR) wherein the peptide, aptamer, synthetic DNA or RNA oligonucleotide or molecular probe, capable of targeting the derivatized RNA molecules to a particular subcellular location in a target cell is linked thereto. In certain embodiments the RNA molecule is an mRNA molecule that encodes a polypeptide. In certain embodiments the UTR is located at the 5′ end of the derivatized RNA molecule. In other embodiments the UTR is located at the 3′ end of the derivatized RNA molecule.

In certain embodiments of the methods provided herein, the derivatized RNA molecules that are targeted to the endoplasmic reticulum of the target cell. Also provided are methods wherein the derivatized RNA molecules that are targeted to the mitochondria of the target cell. Further provided are methods wherein the derivatized RNA molecules that are targeted to the target cell nucleus. In certain embodiments of the provided methods the derivatized RNA comprises a molecular probe capable of targeting the derivatized RNA molecules to a particular subcellular location in a target cell that binds to a target molecule at the targeted subcellular location. In particular embodiments of these methods, the peptide, aptamer, synthetic DNA or RNA oligonucleotide, or molecular probe targets the derivatized RNA molecule to a subcellular location in a recipient cell by recruiting chaperone proteins expressed in the recipient cell that shuttle the derivatized RNA molecule to the subcellular location. In certain particular embodiments of these methods the chaperone proteins are differentially expressed in the target cell. In other embodiments, the peptide, aptamer, synthetic DNA or RNA oligonucleotide, or molecular probe targets the derivatized RNA molecule to a subcellular location in a recipient cell by phase-mediated compartmentalization within the recipient cell.

Further provided are methods for delivering pharmaceutical compositions of the derivatized RNA molecules provided by the invention to cells and tissues wherein the derivatized RNA components of said compositions are directed to specific and particular subcellular targets therein.

In the practice of the methods of the invention the derivatized RNA can be an mRNA encoding a polypeptide, in certain embodiments comprising a therapeutic protein. Polypeptides encoded by derivatized RNA molecules of the invention can include but are not limited to therapeutic proteins produced in vitro using, inter alia, recombinant genetic technologies.

The invention also provides methods for producing derivatized RNA molecules preferably encoding a polypeptide, wherein the RNA molecule further comprises one or more of a peptide, aptamer, synthetic DNA or RNA oligonucleotide or molecular probe, capable of targeting the derivatized RNA molecules to a particular subcellular location in a target cell, the methods comprising the steps of ligating a peptide, an aptamer, a synthetic DNA or RNA oligonucleotide, or a molecular probe to an mRNA molecule encoding a polypeptide, wherein the peptide, the aptamer, the synthetic DNA or RNA oligonucleotide, or the molecular probe targets the derivatized RNA molecule to a subcellular location in a recipient cell. In certain embodiments the methods comprise steps for ligating a synthetic DNA or RNA oligonucleotide to a pre-mRNA molecule; and adding a polyA tail to the DNA or RNA oligonucleotide's 3′ end.

The invention also provides kits for producing the derivatized RNA molecules of the invention, including said RNA molecules, peptide, aptamers, synthetic DNA or RNA oligonucleotides or molecular probes capable of targeting the derivatized RNA molecules to a particular subcellular location in a target cell, and reagents for linking said peptide, aptamers, synthetic DNA or RNA oligonucleotides or molecular probes capable of targeting the derivatized RNA molecules to a particular subcellular location in a target cell, in particular wherein the linkages are covalent linkages. In certain embodiments the derivatized RNA molecules are mRNA molecules encoding a polypeptide, in particular embodiments wherein the polypeptide is a therapeutic protein. Also provided are kits for practicing the methods set forth herein, comprising derivatized RNA molecules, in particular comprising mRNA molecules encoding a polypeptide, wherein the RNA molecule further comprises one or more of a peptide, aptamer, synthetic DNA or RNA oligonucleotide or molecular probe, capable of targeting the derivatized RNA molecules to a particular subcellular location in a target cell, as well as optionally including reagents for introducing the derivatized RNA molecules into a recipient cell. Further provided are kits for achieving delivery of the derivatized RNA molecules of the invention, advantageously as pharmaceutical compositions, to desirable cells and tissues in a human or animal. In all embodiments of the kits provided by this invention instructions for the use of the disclosed methods are also provided as components of such inventive kits.

In a first aspect, the present disclosure provides a derivatized RNA molecule that includes an RNA; and one or more of a peptide, an aptamer, a synthetic DNA or RNA oligonucleotide, or a molecular probe capable of targeting the derivatized RNA molecule to a subcellular location in a recipient cell.

In one embodiment of the first aspect, the RNA is an mRNA encoding a polypeptide.

In one embodiment of the first aspect, the non-coding RNA is a CRISPR guide RNA, an antisense oligonucleotide, an siRNA, or a miRNA.

In one embodiment of the first aspect, the RNA is a non-coding RNA.

In some embodiments, the derivatized RNA molecule of the first aspect or any embodiments thereof further comprises one or a plurality of chemically modified nucleotides.

In one embodiment of the first aspect, the chemically derivatized nucleotides comprise pseudouridine, N1-methylpseudouridine, 5-methylcytidine, or a combination thereof.

In some embodiments, the derivatized RNA molecule of the first aspect or an embodiment thereof further comprises a polyA tail at the 3′ end of the mRNA sequence encoding the polypeptide.

In some embodiments, the derivatized RNA molecule of the first aspect or an embodiment thereof further comprises a eukaryotic cap residue at the 5′ end of the derivatized RNA molecule.

In some embodiments of the first aspect, the peptide, the aptamer, the synthetic DNA or RNA oligonucleotide, or the molecular probe is covalently linked to the derivatized RNA molecule such that translation of the mRNA is not inhibited or reduced.

In some embodiments of the first aspect, the peptide, the aptamer, the synthetic DNA or RNA oligonucleotide, or the molecular probe is covalently linked to the derivatized RNA molecule such that translation of the mRNA is enhanced.

In some embodiments of the first aspect, the subcellular location in the recipient cell is the endoplasmic reticulum.

In some embodiments of the first aspect, the subcellular location in the recipient cell is the mitochondria.

In some embodiments of the first aspect, the subcellular location in the recipient cell is the cell nucleus.

In some embodiments of the first aspect, the peptide, the aptamer, the synthetic DNA or RNA oligonucleotide, or the molecular probe is located in an untranslated region (UTR) of the RNA molecule.

In some embodiments of the first aspect, the peptide, the aptamer, the synthetic DNA or RNA oligonucleotide, or the molecular probe is located in the poly A tail.

In some embodiments of the first aspect, the peptide, the aptamer, the synthetic DNA or RNA oligonucleotide, or the molecular probe targets the derivatized RNA molecule to a subcellular location in the recipient cell by binding to a target molecule at the subcellular location.

In some embodiments of the first aspect, the peptide, the aptamer, the synthetic DNA or RNA oligonucleotide, or the molecular probe targets the derivatized RNA molecule to a subcellular location in a recipient cell by recruiting chaperone proteins expressed in the recipient cell that shuttle the derivatized RNA molecule to the subcellular location.

In some embodiments of the first aspect, the peptide, the aptamer, the synthetic DNA or RNA oligonucleotide, or the molecular probe targets the derivatized RNA molecule to a subcellular location in a recipient cell by phase-mediated compartmentalization within the recipient cell.

In a third aspect, the present disclosure provides a method of targeting an mRNA encoding a polypeptide to a subcellular location in a recipient cell. The method includes a) introducing into a recipient cell a derivatized RNA molecule of any one of the first or second aspects and embodiments thereof; and b) targeting the derivatized RNA molecule to a subcellular location in the recipient cell by a peptide, an aptamer, a synthetic DNA or RNA oligonucleotide, or molecular probe covalently linked to the derivatized RNA molecule.

In a fourth aspect, the present disclosure provides a method of producing a derivatized RNA molecule according to any one of the first or second aspects and embodiments thereof. The method includes ligating a peptide, an aptamer, a synthetic DNA or RNA oligonucleotide, or a molecular probe to an mRNA molecule encoding a polypeptide, wherein the peptide, the aptamer, the synthetic DNA or RNA oligonucleotide, or the molecular probe targets the derivatized RNA molecule to a subcellular location in a recipient cell.

In a fifth aspect, the present disclosure provides a method of producing a derivatized RNA molecule according to any one of the first or second aspects and embodiments thereof. The method includes the steps of: a) ligating a synthetic DNA or RNA oligonucleotide to a pre-mRNA molecule; and b) adding a poly A tail to the DNA or RNA oligonucleotide's 3′ end. In one embodiment, the polypeptide comprises a therapeutic protein. In one embodiment, the therapeutic protein comprises Cystic Fibrosis Transmembrane Conductance Regulator (CFTR).

These and other features, objects, and advantages of the invention will become better understood from the description that follows. In the description, reference is made to the accompanying drawings, which form a part hereof and in which there is shown by way of illustration, not limitation, embodiments of the invention. The description of preferred embodiments is not intended to limit the invention to cover all modifications, equivalents, and alternatives. Reference should therefore be made to the claims recited herein for interpreting the scope of the invention.

### BRIEF DESCRIPTION OF THE DRAWINGS

FIG. 1A through FIG. 1C show a summary of methods for designing and producing mRNA—molecular probe conjugates, wherein FIG. 1A illustrates a first synthetic scheme and alternative second synthetic scheme; FIG. 1B illustrates a molecular mechanism for the derivatized RNA molecules of the invention to enter into a mammalian cell and achieve specific subcellular localizations depending on the nature of the conjugated peptide, aptamer, molecular probe, or NDA or RNA oligonucleotide; and FIG. 1C illustrates modes of probe-mediated mRNA subcellular targeting.

FIG. 2 illustrates two methods for conjugating peptides two mRNA molecules, wherein the mRNA encodes green fluorescence protein (GFP) and the peptide is conjugated to a thiol-derivatized 3′ terminal adenylate residue in a poly A tail covalently linked to the mRNA via a maleimide at the N-terminal amino acid of the peptide, or the peptide, conjugated via maleimide to a thiol-derivatized 3′ terminal adenylate residue in a polyA tail, is conjugated to a free 3′ hydroxyl group of the mRNA.

### DETAILED DESCRIPTION

Provided herewith is a more detailed description of the compositions, methods, and kits comprising the invention, which is provided to explain and enhance but not replace or be a substitute for the claims set forth below.

All publications, including but not limited to patents and patent applications, cited in this specification are herein incorporated by reference as though set forth in their entirety in this application.

### Definitions

As used in the specification, articles “a” and “an” are used herein to refer to one or to more than one (i.e., at least one) of the grammatical object of the article. By way of example, “an element” means at least one element and can include more than one element.

“About” is used to provide flexibility to a numerical range endpoint by providing that a given value can be “slightly above” or “slightly below” the endpoint without affecting the desired result. The term “about” in association with a numerical value means that the numerical value can vary by plus or minus 5% or less of the numerical value.

Throughout this specification, unless the context requires otherwise, the word “comprise” and “include” and variations (e.g., “comprises,” “comprising,” “includes,” “including”) will be understood to imply the inclusion of a stated component, feature, element, or step or group of components, features, elements, or steps but not the exclusion of any other integer or step or group of integers or steps.

As used herein, “and/or” refers to and encompasses any and all possible combinations of one or more of the associated listed items, as well as the lack of combinations where interpreted in the alternative (“or”).

Recitation of ranges of values herein are merely intended to serve as a succinct method of referring individually to each separate value falling within the range, unless otherwise indicated herein. Furthermore, each separate value is incorporated into the specification as if it were individually recited herein. For example, if a range is stated as 1 to 50, it is intended that values such as 2 to 4, 10 to 30, or 1 to 3, etc., are expressly enumerated in this disclosure. These are only examples of what is specifically intended, and all possible combinations of numerical values between and including the lowest value and the highest value enumerated are to be considered to be expressly stated in this disclosure.

As utilized in accordance with the present disclosure, unless otherwise indicated, all technical and scientific terms shall be understood to have the same meaning as commonly understood by one of ordinary skill in the art. Unless otherwise required by context, singular terms shall include pluralities and plural terms shall include the singular.

As used herein, the terms “derivatized” and “functionalized” shall be understood to be equivalent, to the extent that particular embodiments of the derivatized RNA molecules have by benefit of derivatization thereof a function, particularly with regard to subcellular location in a recipient cell.

As set forth herein, this invention is directed to and provides derivatized RNA molecules that can be targeted to one or a plurality of subcellular locations. Conventionally, subcellular mRNA localization as thought to be limited to rare transcripts encoding specific proteins. Recent research has detected widespread occurrence of the phenomenon (see, Holt & Bullock, 2013, Science 326:1212-1216), for example, with up to 70% of mRNA species being asymmetrically distributed in Drosophila embryo cells (Lecuyer et al., 2007, Cell 131:174.) This phenomenon is also seen in vertebrate cells including mammalian fibroblasts.

One of the mechanisms for subcellular localization of mRNA species is localized anchorage. Localization is facilitated inter alia by the presence of cis-acting sequences known as zip codes in the mRNA itself, see Jambhekar & Derisi, 2007, RNA 13:625, frequently found in untranslated regions (Chartrand et al., 2002, Mol. Cell. 10:1319). Intracellular mRNA transport and subcellular localization has been observed (see, Das et al., 2021, Nat. Rev. Molec. Cell Biol. 22:483-504), wherein mRNAs have been observed to be associated with subcellular locations, creating “translation hotspots.” These phenomena have been proposed as means by which cells “fine-tune” their physiology to respond to either intracellular or environmental “cues” and to enable spatial and temporal control of gene expression.

One of the mechanisms for subcellular localization of mRNA species is localized anchorage. Localization is facilitated inter alia by the presence of cis-acting sequences known as zip codes in the mRNA itself, see Jambhekar & Derisi, 2007, RNA 13:625, frequently found in untranslated regions (Chartrand et al., 2002, Mol. Cell. 10:1319). Intracellular mRNA transport and subcellular localization has been observed (see, Das et al., 2021, Nat. Rev. Molec. Cell Biol. 22: 483-504), wherein mRNAs have been observed to be associated with subcellular locations, creating “translation hotspots.” These phenomena have been proposed as means by which cells “fine-tune” their physiology to respond to either intracellular or environmental “cues” and to enable spatial and temporal control of gene expression.

Certain subcellular localization signals are associated with specific nucleotide sequences located, inter alia, 3′ to the coding sequence in an untranslated region; specific examples of such localization signals can be found in nuclear DNA-encoded mitochondrial proteins (Michaud et al., 2014, Proc. Natl Acad. Sci. USA 111: 8991-8996; Vincent et al., 2017, Plant J. 92: 1132-1142). Another example is the use of ribonucleoproteins to transport mRNA in distal dendrites and axons in neuronal cells (Das et al., 2019, Curr. Opin. Neurobiol. 57: 110-116; Holt et al., 2019, Nat. Struct. Mol. Biol. 26: 557-566; Biever et al., 2019, Curr. Opin. Neurobiol. 57: 141-148). Endoplasmic reticulum-localized mRNA has been observed in secretory cells, facilitating secreted protein production from “rough” ER (Walter et al., 1994, Annu. Rev. Cell Biol. 10: 87-119), although this phenomenon has been observed as well with intracellular “housekeeping” proteins such as GAPDH (Masibay et al., 1988, Mol. Cell. Biol. 8: 2288-2294; Stephens et al., 2005, Molec. Biol. Cell 16: 5819-5831; Liao et al., 2011, J. Cell Sci. 124: 589-599).

This application discloses and claims compositions, reagents, methods, and kits comprising derivatized RNA molecules preferably encoding a polypeptide, wherein the RNA molecule further comprises one or more of a peptide, aptamer, synthetic DNA or RNA oligonucleotide or molecular probe, capable of targeting the derivatized RNA molecules to a particular subcellular location in a target cell. mRNAs derivatized according to the reagents and methods set forth herein advantageously are those encoding proteins including membrane-anchored proteins (see, e.g., Kinoshita, 2020, Open Biology 10: 190290); C-tail anchored proteins (see, e.g., Borgese & Fasana, 2011, Biochim Biophys Acta 1808: 937-946; Pauick & Bertozzi, 2008, Biochemistry 47: 6991-7000); and secreted proteins (see, e.g., Nalbant et al., 2005, BMC Genomics 6: 11; Melkonyan et al., 1997, Proc. Natl. Acad. Sci. USA 94: 13636-13641; Wallis, 2009, Growth Hormone & IGF Res. 19: 12-23; Mahiab & Linial, 2014, PloS Omput. Biol. 10: e1003294); and Das et al., 2021, Nature 22: 483-504 for review).

As used herein, mRNAs functionalized with small molecules, aptamers, or peptides (i.e. “molecular probes”) will be understood to have altered subcellular distribution. Many molecular probes, such as small molecules, are available that target specific subcellular locations, including but not limited to mitochondria, endoplasmic reticulum (ER), plasma membrane, lysosome, cytoskeleton, cell-cell junction, centromere, mitotic spindles, and include asymmetric or heterogenous distribution that mimics natural mRNAs; see, Gao et al., 2019, Fluorescent probes for organelle-targeted bioactive species imaging, Chemical science 10: 6035-6071. Functionalizing therapeutic mRNAs with small molecules, aptamers, peptides, or derivatives thereof as provided herein alter mRNA subcellular localization. Because specification of subcellular locations of derivatized RNA molecules, particularly mRNA molecules encoding a polypeptide and in particular a therapeutic protein are programmable based on the nature of molecular probes used according to the methods disclosed herein, improved beneficial properties of therapeutic mRNA can be achieved.

In further embodiments, the reagents and methods provided herein can advantageously direct mRNAs to the endoplasmic reticulum for secreted proteins used for therapeutic purposes. Polypeptides encoded by derivatized RNA molecules of the invention can include but are not limited to therapeutic proteins produced in vitro using, inter alia, recombinant genetic technologies (see, e.g., Dalton & Barton, 2014, Prot. Sci. 23: 517-525; Bonin-Debs et al., 2005, Exp. Opin. Biol. Ther. 4: 551-558; Aydin et al., 2012, JoVE Biol. 65: e44041).

Derivatized RNA can be used to facilitate localized and rapid translation of a number of critical and/or medicinally important proteins. Such proteins include by are not limited to insulin, human growth factor, pramlintide acetate, pegvisoman, mecasermin, Factor VIII, Factor IX, Protein C concentrate, Erythropoietin, Filgrastim, sargrasmostim 36 and 37, oprelvekin, human follicle-stimulating hormone, human chorionic gonadotropin, human luteinizing hormone, interleukin 2, Thyroid hormone, parathyroid hormone, adenosine deaminase, SMN, MECP2, IFNα2a, IFNα2b, IFNαn3, rIFN-β, IFNγ, calcitonin, somatostatin, human bone morphogenic protein 2, gonadotropin-releasing hormone, keratinocyte growth factor, platelet-derived growth factor, human B-type natriuretic peptide, hirudin, and the like. A person of ordinary skill in the art would recognize that many diseases possess an underlying pathophysiology attributable to misfolded or absent proteins as a result of mutated, dysfunctional, or missing genes. As such, derivatized RNA can be used to deliver ready-to-translate RNA to cells and cellular components in need of a missing or functional protein.

In yet other embodiments of this invention, derivatized RNA can be an interfering RNA (RNAi). A person of ordinary skill in the art understands that RNAi is an RNA molecule that can be involved in sequence-specific suppression of gene expression by forming double stranded-RNA that either blocks translation of an mRNA or results in cleavage of an mRNA. See Setten et al., The current state and future directions of RNAi-based therapeutics, Nature Revs. Drug Discovery, 2019, 18:421-446. In one embodiment, the RNAi can be between 15 and 30 bp, and can form duplexes to interact with Dicer enzyme for cleavage and handoff to RNA-induced silencing complex (RISC). In yet another embodiment, the RNAi can be less than 15 bp long. In Yet other embodiments, the RNAi can be greater than 30 bp in length. Alternatively, the derivatized RNA can bypass Dicer and enter the RISC by directly interacting with TAR RNA-binding protein (TARB).

In additional embodiments, the derivatized RNA comprises an RNAi (or siRNA) with chemically modified base positions to reduce degradation by serum nucleases and reduce immunogenic toxicity. In a further embodiment, the derivatized RNA comprises an RNAi that has been chemically ligated to a multivalent GalNAc ligand to form a GalNAc-siRNA complex. In a further embodiment, the derivatized RNA comprises an RNAi that has been coupled with a phospotidylserine (PS) backbone to the two 5′ terminal nucleotides on each strand of the siRNA. In a further embodiment, the derivatized RNA comprises an RNAi that has been chemically modified with a 2′-O-methyl modification on both strands of the siRNA.

In yet other embodiment of the present invention, derivatized RNA can be a double stranded RNA (dsRNA). The dsRNA can be used to trigger de novo DNA methylation through the process of transcriptional gene silencing. Alternatively, the derivatized RNA can comprise a small activating RNA (saRNA), dependent on Ago2 to express certain genes.

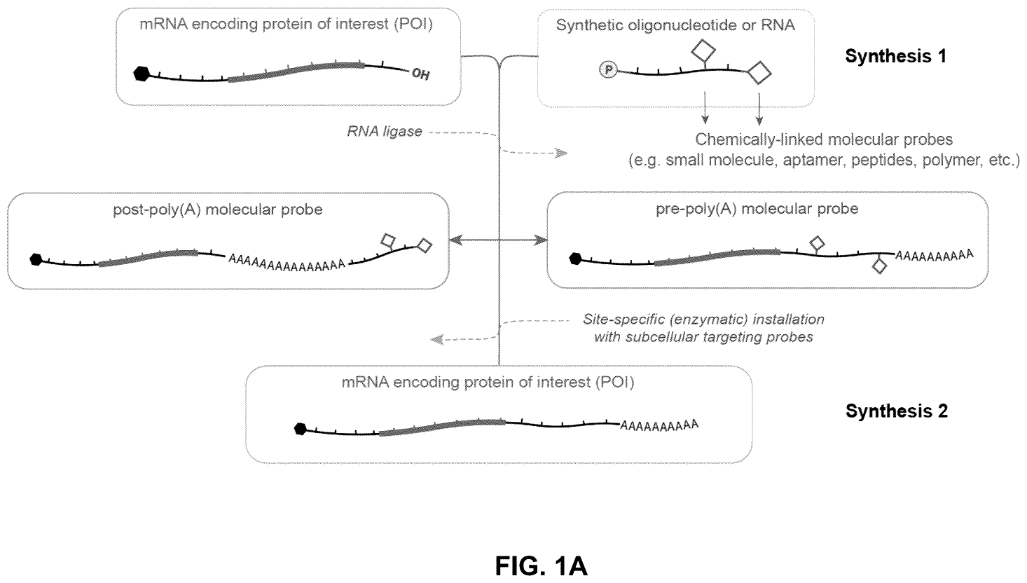

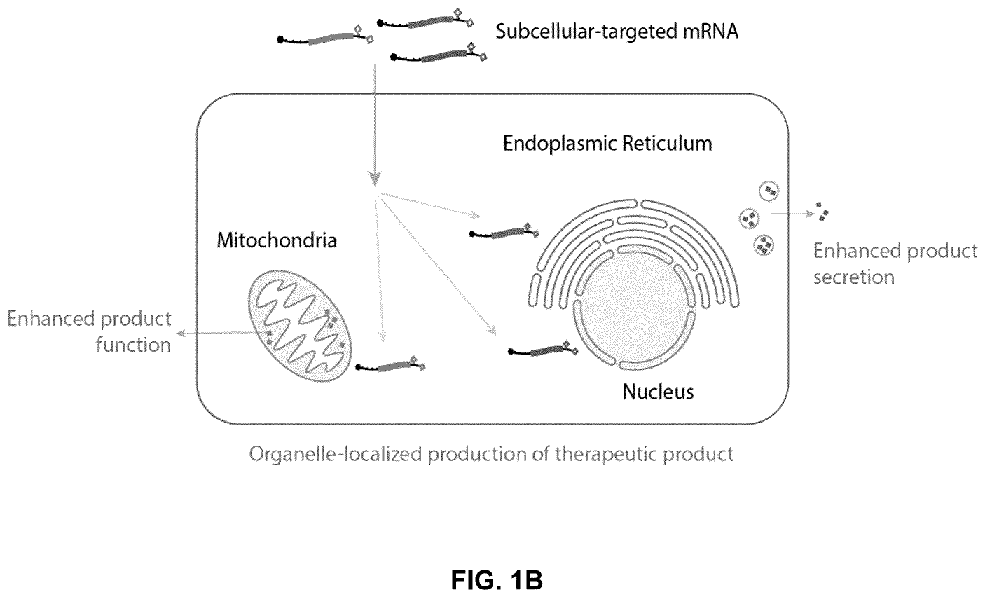

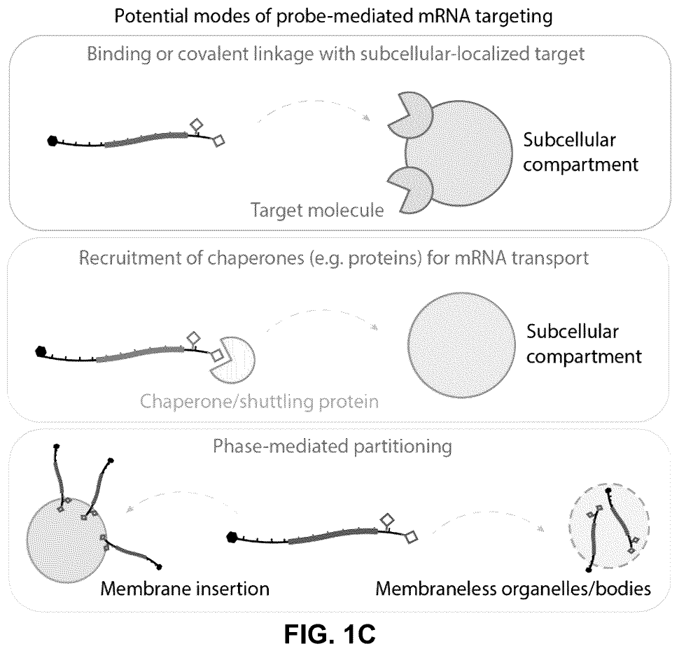

The mRNA-molecular probe conjugates of the invention comprise linear, chemically modified mRNA that encodes any protein of interest (POI), particular therapeutic proteins, having one or more molecular targeting probes conjugated thereto. Conjugation can be designed and achieved depending on the nature of the derivative, the desired subcellular localization, and the size and complexity of the modified mRNA at the 3′ end of the mRNA, particularly the poly A tail, or internally. The POI can contain a signal sequence (N-, C-, or internal) that enables translocation across the membrane of the organelle of interest. Site-specific or region-specific chemical modification of the mRNA is advantageously limited to non-coding regions such as untranslated (UTR) regions (located at the 5′ or 3′ end of the coding sequence, or both the 5′ and 3′ end thereof) and/or the poly (A) tail, as chemical modification of the mRNA protein coding region (CDS) could impair ribosomal decoding. Eukaryotic mRNA advantageously contains a cap and poly(A) tail to enable efficient cap-dependent translation in cells (illustrated in FIG. 1a). Subcellular targeting enables therapeutic proteins to be expressed at higher levels in their native context, potentially enabling higher protein activity, enhanced co-translational folding, or higher levels of secretion (illustrated in FIG. 1b).

Molecular probes can be small molecules, target-binding aptamers, peptides, or other polymers of interest. Furthermore, molecular probes can cause mRNA subcellular partitioning through a variety of mechanisms. Some common targeting mechanisms can include: (1) direct binding to target molecule(s) in the subcellular location of interest; (2) recruitment of chaperones (e.g. proteins) that shuttle mRNAs to cellular compartments; (3) phase-mediated compartmentalization, such as molecular probe insertion into plasma membranes or recruitment to membraneless organelles/cellular compartments (illustrated in FIG. 1c)

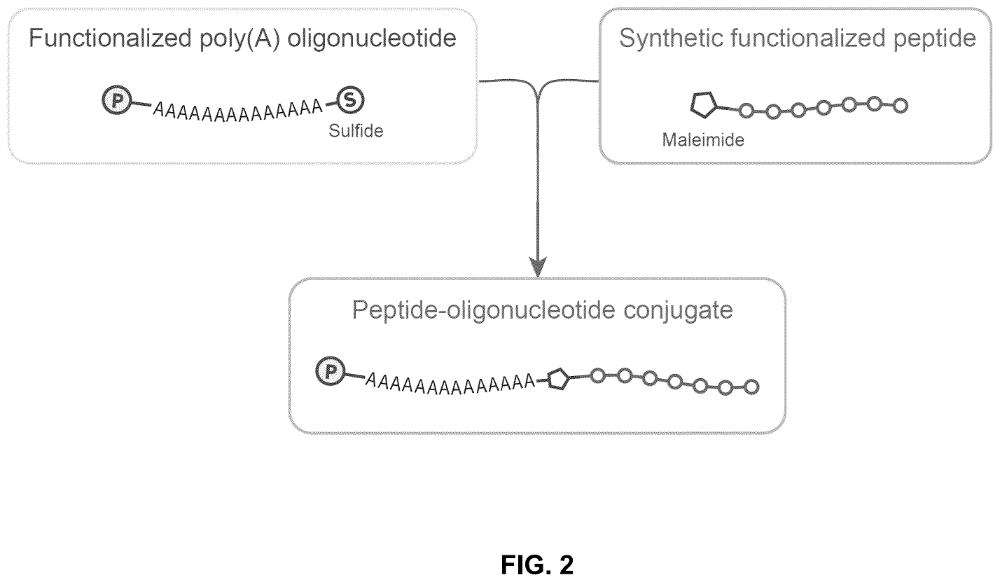

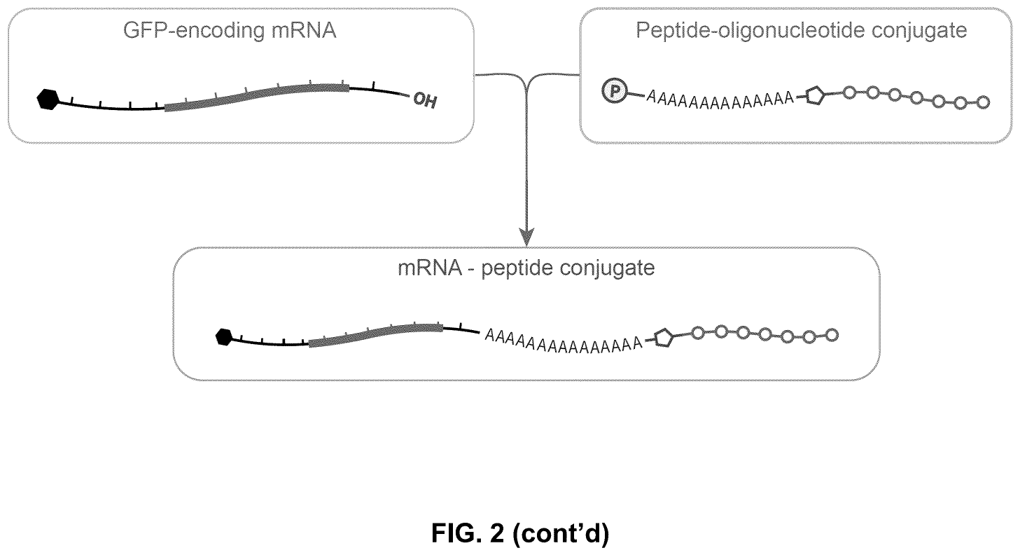

FIG. 2 shows a synthetic scheme for mRNA-peptide conjugates, where the peptides are naturally occurring subcellular localization sequences, or aptamers designed to bind a specific cellular target. Synthesis begins with the reaction of a 3′ click-functionalized poly(rA) oligo mimicking the poly(A) tail, to an N-terminal click-functionalized peptide of interest (FIG. 2a); see, e.g., DeForest et al., Chem, Mater. 22: 4783-4790; and generally. Deng et al., 2020, Nat. Comm. Chem. 3:67). This peptide-oligonucleotide conjugate is then ligated to the 3′ terminus of an mRNA to generate a 3′ peptide functionalized mRNA (FIG. 2b).

### Subcellular Targets

The derivatized RNA compositions disclosed herein advantageously can be targeted to specific intracellular organelles.

### Mitochondria

In one embodiment of the invention, derivatized RNA molecules are directed towards targeting subcellular locations associated with the mitochondria. Specifically, derivatized RNA are used in the treatment of mitochondrial associated diseases.

Mitochondrial associated diseases include but are not limited to the following, Autosomal Dominant Optic Atrophy, Alpers Disease, Barth Syndrome, Beta-Oxidation defects [such as LCAD, LCHAD, MAD, MCAD, SCAD, SCHAD, VLCAD], Carnitine-Acyl-Carnitine Deficiency, Carnitine deficiency, Complex I deficiency, Complex II deficiency, Complex III deficiency, Complex IV deficiency, COX deficiency, CPT deficiency, Creatine deficiency syndromes [further comprising Guanidinoaceteate Methyltransferase Deficiency (GAMT Deficiency), L-Arginine: Glycine Amidinotransferase Deficiency (AGAT Deficiency), and SLC6A8-Related Creatine Transporter Deficiency (SLC6A8 Deficiency)], Co-Enzyme Q10 Deficiency, CPEO, CPT II deficiency, Lactic Acidosis, LBSL-Leukodystrophy, LCA deficiency, LCHA deficiency, Complex V deficiency (T8993g or T8993C), neuropathy ataxia and retinitis pigmentosa (NARP), Leigh Disease/syndrome, Leber Hereditary Optic Neuropathy, Luft Disease, MAD/Glurtaric Aciduria Type II, medium-chain Acyl-CoA dehydrongenase deficiency (MCAD), myoclonic epilepsy and ragged-red fiber disease (MERRF), mitochondrial encephalomyopathy lactic acidosis and stroke-like episodes (MELAS), mitochondrial enoyl CoA reductase protein associated neurodegeneration (MEPAN), mitochondrial recessive ataxia syndrome (MIRAS), Mitochondrial DNA Depletion, Mitochondrial Encephalopathy, myoneurogastrointestinal disorder and encephalopathy (MNGIE), Neuropathy, and Pearson Syndrome or Kearns-Sayre Syndrome.

Additional mitochondrial associated diseases can also be characterized by POLG mutations and their associated pathologies including but not limited to the following: Alpers-Huttenlocker syndrome (AHSD), myocerebrohepatopathy spectrum (MCHS), myoclonic epilepsy myopathy sensory ataxia (MEMSA) and previously described spinocerebellar ataxia with epilepsy (SCAE), autosomal recessive progressive external ophthalmoplegia (arPEO), chronic progressive external ophthalmoplegia plus (CPEO+).

Other mitochondrial associated diseases include but are not limited to Pyruvate Carboxylase Deficiency, PDC deficiency, sensory ataxia neuropathy dysarthria and ophthalmoplegia (SANDO), Short-Chain Acyl-CoA Dehydrogenase Deficiency (SCAD), hyperinsulinism due to short chain 3 hydroxylacyl-CoA dehydrogenase (SCHAD), TK2/myopathic form, and very long-chain acyl-CoA dehydrogenase deficiency (VLCAD).

### Rough Endoplasmic Reticulum

Researchers have demonstrated that endoplasmic reticulum dysfunction can play a role in multiple underlying human pathologies. See Roussel et al., Endoplasmic reticulum dysfunction in neurological disease, Lancet Neurol. 2013 January; 12(1): 105-18; Ozcan and Tabas, Role of Endoplasmic Stress in Metabolic Disease and Other Disorders, Annu. Rev. Med. 2012, 63: 317-328;

In one embodiment of the invention, derivatized RNA molecules can be directed to target subcellular locations associated with the endoplasmic reticulum for the treatment of neurological disorders such as cerebral ischemia, sleep apnea, Alzheimer's disease, multiple sclerosis, amyotrophic lateral sclerosis, prion diseases, and familial encephalopathy with associated neuroserpin inclusion bodies.

In other embodiments of the invention, derivatized RNA molecules can be directed to target subcellular locations associated with the endoplasmic reticulum for the treatment of Type 2 diabetes and improperly folded ATF6 protein, atherosclerosis, nonalcoholic fatty liver disease and associated SREBP-1c, IRE1-XBP1 pathway suppression, and Alcoholic liver disease.

In yet other embodiments of the invention, derivatized RNA molecules can be used to increase the expression of ER chaperones such as Grp78 and Grp94 to ameliorate unfolded protein response.

### Golgi Apparatus

Researchers have identified Golgi dysfunction to contribute to neurodegenerative and other infections disease pathologies. See, Liu et al., The role of the Golgi apparatus in Disease (Review), Int. J. Mol. Med. 2021 April; 47(4): 38;

In yet other embodiments of the invention, derivatized RNA molecules are used to treat structural and function changes of the Golgi apparatus. Specifically, derivatized RNA molecules are employed as a therapy to alleviate Golgi dysfunction resulting from various membrane trafficking pathways and their associated Golgi resident proteins.

Furthermore, derivatized RNA molecules are used to treat defective proteins, with roles associated towards anterograde trafficking such as GM130, Giantin, Fukutin, Dymeclin and SCYL1BP1.

Additionally, derivatized RNA molecules are used to treat defective proteins, with roles associated with retrograde trafficking such as COGs. Derivatized RNA molecules are also used to treat defective proteins, with roles associated with transport to the endoplasmic reticulum such as TRAPPC2 and GMAP-210.

Derivatized RNA molecules can further be used to treat protein dysfunctions associated with FGD1, ATP2C1, ARFGEF2, DENND5A and BICD.

Furthermore, derivatized RNA employed to treat Golgi dysfunction are used to ameliorate or treat Golgi dysfunction associated diseases such as but not limited to: pulmonary artery hypertension, neurodegenerative diseases, ischemic stroke, arrhythmia, dilated cardiomyopathy, heart failure, hepatitis, liver cirrhosis, hepatocellular carcinoma, congenital muscular dystrophy, Hailey-Hailey disease, Cutis laxa type II, gastric cancer, colon cancer, osteoporosis, skeletal dysplasia, X-linked mental retardation syndrome, Hermansky-Pudlak syndrome, Menkes disease, Wilson's disease, spinocerebellar ataxia type 2, CDG-type II, COPA syndrome, epileptic encephalopathy, Dyggve-Melchior-Clausen syndrome, Aarskog-Scott syndrome, Limb girdle muscular dystrophy, FCMD, progressive myoclonus epilepsy, idiopathic intellectual disability, congenial muscular dystrophy, autosomal dominant nonsyndromatic hearing loss, Smith-McCort dysplasia, Gerodermia osteodyspastica, CDG-II, CDG, and progressive cerebello-cerebral atrophy type 2.

Specifically, derivatized RNA is employed to treat Golgi dysfunction by replacing proteins translated from defective or mutated copies of genes known to cause Golgi dysfunction. Genes known to cause Golgi dysfunction, if defective, include but are not limited to AP1S2, AP3D1, ARFGEF2, ATP2C1, ATP6VIA, ATP6VIE1, ATP6VOA2, ATP7A, ATP7B, ATXN2, Bicaudal-D, COG, COPA, DENND5A, DYM, FGD1, FKRP, Fukutin, GOSR2, HERCI, LARGE, OSPBL2, RAB33B, RAB39B, SIP, SCYLIBP1, SLC35A1, SLC35A2, TMEM165, TRAPPC11, TRAPPC2, TRIP11, and VPS53.

### Cytoskeleton

Researchers have identified cytoskeletal protein aberrations as an underlying contributor to various disease pathologies. See, Ramaekers & Bosman., The cytoskeleton and disease, J. Pathol., 2004, 204(4): 351-354; Lane & McLean, Keratins and skin disorders, J. Pathol., 2004, 204: 367-376; Owens & Lane, Keratin mutations and intestinal pathology, J. Pathol., 2004, 204: 377-385; Clarkson et al., Congenital myopathies: diseases of the actin cytoskeleton, J. Pathol., 2004; 204: 407-417.

In yet other embodiments of the invention, derivatized RNA molecules are used treat cytoskeletal protein aberrations. Specifically diseases associated with improperly folded protein forms or mutations in the genes encoding, α-actin, α- and β-Tropomyosin, Troponin-T, Tubilin, lamin-associated proteins, Septin and Nebulin can be ameliorated with derivatized RNA molecules. In further embodiments of the inventions, derivatized RNA molecules are employed to treat disease pathologies associated with dysfunction of cytoskeletal proteins including but not limited to acidic keratins, basic keratins, Vimentin, Desmin and Desmin associated proteins such as alpha-B-crystallin, acidic glial fibrillary proteins, GFAP, Peripherin, Synemin, NF-L, NF-M, Nestin, α-Internexin, Syncoilin, nuclear lamins, Phakinin, and Filensin.

Furthermore, derivatized RNA employed to treat cytoskeletal protein dysfunction can be used to ameliorate or treat cytoskeletal protein dysfunction associated diseases such as but not limited to: neoplasia, neurodegenerative diseases, tauopathies and α-synucleopathies, Alexander disease, fibrotic pulmonary disease, immotile cilia syndrome, Wiskott-Aldrich Syndrome (WAS), Charcot-Marie-Tooth disease, amyotrophic lateral sclerosis, congenital myopathy, cardiovascular disease syndromes, inflammatory bowel disease, liver cirrhosis chronic pancreatitis, along with cancer and other hyper proliferative diseases.

### Lysosome

Researchers have identified aberrations in lysosomal proteins resultant form single-gene defects to contribute to a significant number of lysosomal storage disorders and associated human diseases. See Rajkumar V, Dumpa V. Lysosomal Storage Disease. [Updated 2022 Jan. 28]. In: StatPearls [Internet]. Treasure Island (FL): StatPearls Publishing; 2022 January-. Available from: www.ncbi.nlm.nih.gov/books/NBK563270/.

In yet other embodiments of the invention, derivatized RNA molecules are used treat lysosomal storage disorders including but not limited to: Maroleaux-Larry Syndrome, Sly Syndrome, Neuronal Ceroid lipfluscinosis NCL 1 through 14, Galaclosialidosis, infantile sialic acid storage disease, Salla disease, Sialuria, Sialisosis I and II, Mucolipidosis I, I-Cell disease (Mucolipidosis II), Pseudo-Hurler-Polydystrophy (Mucolipidosis III), Mucolipidosis IV, Lysosomal Acid lipase deficiency infantile and childhood types, Pompe's disease (Glycogen storage disease type II), Danon disease, and Cystinosis.

Specifically, in certain embodiments of the present invention, derivatized RNA molecules are used to treat diseases associated with improperly folded proteins known to contribute to lysosomal storage disorder, the proteins including but not limited to: N-acetylgalactosamne-4-sulfate, Beta glucuronidase, catabolic enzymes, protective proteins and Cathepsin A, Sialin, Uridinediphosphate-N-acetylglucosamine 2-epimerase, Neuraminidase 1 (NEU1), GlcNAc-1-phosphostansferase, Lucolipin-1, lysosomal acid lipase, Acid alpha-glucosidase (GAA), LAMP2 protein, and Cystinosin.

In preferred embodiments of the present invention, derivatized RNA molecules are used treat lysosomal storage disorders by encoding for proteins to replace those by defective or insufficient transcription of at least the following genes: Aryl Sulfatase B, GUSB, CLN1-CLN14, CTSA, SLC17A5, GNE, NEU1, GNTAB, MCOLNI, LIPA, GAA, LMAP2, and CTNS.

### Centriole

Researchers have asserted that centrioles play a key role in auto-degradation of defective cellular machinery in a process known as centrosome-phagy. See Wu et al., Centrosome-phagy: implications for human diseases, Cell & Biosciences, 2021, 11(49).

In one embodiment of the present invention, derivatized RNA molecules are used to treat human disorders associated with defective or insufficient translation of at least the following proteins, OFD1, PCMI, Cep55, Cep63, Cep131, PDK1, and PDK2. Defects in at least these proteins associates with the centrosome contribute to ciliopathies, ageing, and various cancers including but not limited to thyroid cancer, renal cancer, breast cancer, ovarian cancer, and colon cancer.

In yet other embodiments of the invention, derivatized RNA molecules are used to treat ciliopathies including but not limited to polycystic kidney disease, nephronophthisis, retinitis pigmentosa, Bardet-Biedl syndrome, Joubert syndrome, and Meckel syndrome. Specifically, derivatized RNA molecules are used to treat mutations or down regulations in at least the proteins PCM1, BBS4, Cep290, and/or OFD1.

In yet a further embodiment of the invention, derivatized RNA molecules are used to treat the effects of aging and aging-related diseases. Specifically, derivatized RNA molecules are used to treat mutations or down regulations in at least the proteins NEDD1, Cep192, and Percentenrin.

In alterative embodiments, the invention provides derivatized mRNA molecules encoding therapeutic proteins for acute therapy in a human or animal (including but not limited to tissue plasminogen activator (tPA), erythropoietin (EPO), growth hormone (hGH) and the like). In other alternative embodiments the invention provides derivatized mRNA molecules encoding proteins for replacing or restoring products of genes dysfunctional in a human or animal (for example, adenosine deaminase, hexosaminidase-A, human beta-globin) which have traditionally been targets for gene therapy. The derivatized mRNA molecules of the invention provide an alternative that requires maintenance administration in contrast to the (generally unrealized) goals of gene therapy, but such maintenance therapies have been used to successfully address many diseases (diabetes, for example) and this use of the derivatized mRNA molecules of this invention avoids many of the disadvantages associated with convention gene therapy approaches to treating genetic disease. Yet alternative embodiments of the derivatized mRNA molecules of this invention encode immunogens from viruses, bacteria, fungi, or parasites that can be targeted, inter alia, to antigen-presenting cells to provide more effective, RNA-based vaccines. One particular embodiment of the derivatized mRNA molecules of this invention encode the cystic fibrosis Transmembrane Conductance Regulator (CFTR) (see, Kim & Skach, 2012, Front. Pharmacol. 3: 201).

Pharmaceutical Compositions for Delivery and Methods Therefor

The present invention provides pharmaceutical compositions comprising derivatized RNA molecules of the invention. In certain embodiments, pharmaceutical compositions of the invention further comprise pharmaceutically acceptable excipients and in certain other embodiments comprise one or more additional therapeutics agents.

In some embodiments, the compositions are suitable to be administered to a human subject in need thereof. In the context of the present disclosure, “active ingredient” refers generally to the derivatized RNA molecules described herein as well as any additional therapeutic agents provided therewith.

It is generally understood by a person of ordinary skill in the art that the compositions described herein are also suitable for administration to any non-human subjects as well. A person of ordinary skill in the veterinary arts will understand that pharmaceutical compositions described herein can be suitable for administration to mammals including but not limited to primates, cattle, pigs, horses, sheep, goats, cats, dogs, mice, rats, whales, and other mammals. A person of ordinary skill in the veterinary arts also will understand that pharmaceutical compositions described herein can be suitable for administration to birds including by not limited to chickens, ducks, geese, turkey, and other domesticated birds, as well as wild birds particularly endangered species of such birds. Additionally, a person of ordinary skill in the veterinary arts will understand that pharmaceutical compositions described herein can be suitable for administration to a wide variety of fish including commercial or wild salmon, tuna, cod, sardine, zebra fish, shark, or the like.

Pharmacological compositions described herein can be prepared by any method known or developed in the art of pharmacology, immunology, virology, or in biotechnology in general.

In some embodiments, the formulations of a pharmacological composition described herein can comprise a unit dose of at least one derivatized RNA, in addition to at least one other pharmaceutically acceptable excipient. Such excipients can include but are not limited to, solvents, dispersions, buffers, diluents, surfactants, emulsifiers, isotonic agents, preservatives, thickeners, lubricating agents, oils, or the like.

In some embodiments, the pharmacological composition can comprise a delivery mechanism further comprising a lipid nanoparticle. The size of the lipid nanoparticle can be altered to counteract immunogenic response from the subject, or to allow for increased potency and pharmacological activity.

In other embodiments, the pharmacological composition can comprise a delivery mechanism further comprising a lipidoid as previously described in the art. See Akinc et al., Nat Biotechnol. 2008 26:561-596; Frank-Kamenetsky et al., Proc Natl Acad Sci USA. 2008 105:11915-11920; Akinc et al., Mol Ther. 2009 17:872-879; Love et al., Proc Natl Acad Sci USA 2010 107:1864-1869; Leuschner et al., Nat Biotechnol. 2011 29:1005-1010, all of which is incorporated herein in their entirety. Lipidoids refers broadly to lipid nanoparticles, liposomes, lipid emulsions, lipid micelles and the like. Lipidoids containing the pharmacological composition comprising the derivatized RNA can be administered parenterally by means including but not limited to, intravenous injection, intramuscular injection, subcutaneous injection, via dialysate, intrathecal injection, or intracranial injection.

A person of ordinary skill in the art would also recognize that other nucleotide delivery mechanisms exist such as the use of viral like, or viral derived particles. See Rohovie et al., Bioengineering & Translational Med., 2016 2(1): 43-57. Virus like particles can include coat proteins or viral capsids of a virus. Such particles can be PEGylated or further annealed to compounds that avoid phagocytotic clearance. Additionally, the surface of the virus like particle can be further functionalized to provide cellular specific targeting, facilitate extravasation, facilitate radio labeling, improve permeability across cellular boundaries, or to transcytose the blood-brain barrier. The virus like particles can be derived for animal viruses, bacteriophages, or plant viruses. Examples of suitable virus for derivation of a virus like particle delivery mechanism include but are not limited to cowpea chlorotic mottle virus, cowpea mosaic virus, hepatitis B virus (core), enterobacteria phage MS2, Salmonella typhimurium P22, enterobacteria phage Qβ amongst other suitable viruses. Derivatized RNA payloads can be loaded into the virus like particles by electrostatic adsorption or any other suitable method known to a person of ordinary skill in the art.

Various exemplary embodiments of compositions and methods according to this invention are now described in the following non-limiting Examples. The Examples are offered for illustrative purposes only and are not intended to limit the scope of the invention in any way. Indeed, various modifications of the invention in addition to those shown and described herein will become apparent to those skilled in the art from the foregoing description and the following examples and fall within the scope of the appended claims.

### EXAMPLES

mRNA-molecular probe conjugates are designed as follows and set forth in FIG. 1. For mRNA functionalization via ligation of modified oligos or RNA fragments, modified oligonucleotides containing reactive groups are functionalized with molecular probes (e.g. small molecules, aptamers, or peptides targeting proteins or subcellular locations/organelles). These oligonucleotides are then ligated to mRNAs in such a way that translational efficiency is not reduced. This modification strategy can contain one or more molecular probes to enhance avidity to target sites. The modifications can also be ligated to the 5′ end or at an internal sequence, provided they do not interfere with translation. See FIG. 1a1. Alternatively, as shown in FIG. 1a2, subcellular targeting probes are enzymatically attached to an mRNA using nucleic acid-modifying enzyme s (such as T4 RNA ligase, T4 DNA ligase, DNA or RNA ligating ribozymes)

The practice of the invention is illustrated in FIG. 1b. Subcellular targeted RNA species, comprising specific peptides, aptamers, DNA or RNA oligonucleotides, or molecular probes are introduced into a cell (wherein a mammalian cell is illustrated) and specifically transported to the endoplasmic reticulum, cell nucleus, or mitochondria, with phenotypic effects specific for each subcellular target. Therapeutic mRNA-probe conjugates can localize to organelles to enable higher expression of a protein of interest (POI) in a relative subcellular context. This strategy can theoretically be applied to any subcellular location, including but not limited to membrane-bound organelles.

FIG. 1c illustrates potential modes of molecular probe targeting mechanisms. Probe targeting can be mediated by specific binding to organelle-localized targets; recruitment of shuttling cofactors; or phase separation into membranes or membraneless subcellular structures.

A synthetic scheme for producing an mRNA-peptide aptamer conjugates is set forth in FIG. 2. A ribonucleotide poly (A) oligonucleotide containing a 5′ ligatable end and 3′ click functional group (IDT format:/5Phos/rArArArArArArArArArArArArArArArArArA rA*rA*rA* rA*rA*rA*/3ThioMC3-D/), is provided having with internal phosphorothioate groups to increase nuclease-resistance from Integrated DNA Technologies (Coralville, IA; IDT). A synthetic peptide containing a click chemical group (Genscript: Maleimide-YRMQLLSCIALSLALVTNS (SEQ ID NO: 1), or Maleimide-FVFLVLLPLVS (SEQ ID NO: 2)) was used, wherein the click-reactive peptide sequences are derived from IL-2 signal sequence, or the SARS-COV-2 spike protein signal sequence, respectively, which are endoplasmic reticulum (ER)-targeting sequences; see, Zhang et al., 2005, Alteration in the IL-2 signal peptide affects secretion of proteins in vitro and in vivo, J Gene Med. 7: 354-65 and Huang et al., 2020, Structural and functional properties of SARS-COV-2 spike protein: potential antivirus drug development for COVID-19, Acta Pharmacologica Sinica 41: 1141-1149. Other signal sequences have also been identified for alternative subcellular organelle targeting. Conventional RNA ligases (e.g. T4 RNA ligase) are used for ligation of oligo-peptide conjugate to mRNA at the 3′ terminus to product mRNAs containing 3′ peptide aptamers will be synthesized.

Those of skill in the art will recognize or be able to ascertain using no more than routine experimentation, many equivalents to the specific embodiments of the invention described herein. Such equivalents are intended to be encompassed by the following claims.

1. A derivatized RNA molecule, comprising:
an RNA; and
one or more of a peptide, an aptamer, a synthetic DNA or RNA oligonucleotide, or a molecular probe capable of targeting the derivatized RNA molecule to a subcellular location in a recipient cell.

2. The derivatized molecule according to claim 1, wherein the RNA is an mRNA encoding a polypeptide.

3. The derivatized molecule according to claim 1, wherein the RNA is a non-coding RNA.

4. The derivatized molecule according to claim 3 wherein the non-coding RNA is a CRISPR guide RNA, an antisense oligonucleotide, an siRNA, or a miRNA.

5. The derivatized RNA molecule of claim 1 further comprising one or a plurality of chemically modified nucleotides.

6. The derivatized RNA molecule of claim 5, wherein the chemically derivatized nucleotides comprise pseudouridine, N1-methylpseudouridine, 5-methylcytidine, or a combination thereof.

7. The derivatized RNA molecule of claim 2 further comprising a polyA tail at the 3′ end of the mRNA sequence encoding the polypeptide.

8. The derivatized RNA molecule of claim 1 further comprising a eukaryotic cap residue at the 5′ end of the derivatized RNA molecule.

9. The derivatized RNA molecule of claim 2, wherein the peptide, the aptamer, the synthetic DNA or RNA oligonucleotide, or the molecular probe is covalently linked to the derivatized RNA molecule such that translation of the mRNA is not inhibited or reduced.

10. The derivatized RNA molecule of claim 2, wherein the peptide, the aptamer, the synthetic DNA or RNA oligonucleotide, or the molecular probe is covalently linked to the derivatized RNA molecule such that translation of the mRNA is enhanced.

11. The derivatized RNA molecule of claim 1, wherein the subcellular location in the recipient cell is the endoplasmic reticulum.

12. The derivatized RNA molecule of claim 1, wherein the subcellular location in the recipient cell is the mitochondria.

13. The derivatized RNA molecule of claim 1, wherein the subcellular location in the recipient cell is the cell nucleus.

14. The derivatized RNA molecule of claim 1, wherein the peptide, the aptamer, the synthetic DNA or RNA oligonucleotide, or the molecular probe is located in an untranslated region (UTR) of the RNA molecule.

15. The derivatized RNA molecule of claim 7, wherein the peptide, the aptamer, the synthetic DNA or RNA oligonucleotide, or the molecular probe is located in the polyA tail.

16. The derivatized RNA molecule of claim 1, wherein the peptide, the aptamer, the synthetic DNA or RNA oligonucleotide, or the molecular probe targets the derivatized RNA molecule to the subcellular location in the recipient cell by binding to a target molecule at the subcellular location.

17. The derivatized RNA molecule of claim 1, wherein the peptide, the aptamer, the synthetic DNA or RNA oligonucleotide, or the molecular probe targets the derivatized RNA molecule to the subcellular location in the recipient cell by recruiting chaperone proteins expressed in the recipient cell that shuttle the derivatized RNA molecule to the subcellular location.

18. The derivatized RNA molecule of claim 1, wherein the peptide, the aptamer, the synthetic DNA or RNA oligonucleotide, or the molecular probe targets the derivatized RNA molecule to the subcellular location in the recipient cell by phase-mediated compartmentalization within the recipient cell.

19. A method of targeting an mRNA encoding a polypeptide to a subcellular location in a recipient cell, comprising:
a) introducing into the recipient cell the mRNA encoding the polypeptide, wherein the mRNA is derivatized by a covalent linkage to a peptide, an aptamer, a synthetic DNA or RNA oligonucleotide, or a molecular probe capable of targeting the mRNA to the subcellular location in the recipient cell; and
b) targeting the the mRNA to the subcellular location in the recipient cell.

20. A method of producing a derivatized RNA molecule according to claim 1, comprising:
ligating the peptide, the aptamer, the synthetic DNA or RNA oligonucleotide, or the molecular probe to the RNA, wherein the RNA is an mRNA molecule encoding a polypeptide,
wherein the peptide, the aptamer, the synthetic DNA or RNA oligonucleotide, or the molecular probe targets the derivatized RNA molecule to the subcellular location in the recipient cell.

21. A method of producing a derivatized RNA molecule comprising the steps of:
a) ligating a synthetic DNA or RNA oligonucleotide to a pre-mRNA molecule to produce the derivatized RNA molecule; and
b) adding a polyA tail to the synthetic DNA or RNA oligonucleotide's 3′ end,
wherein the synthetic DNA or RNA oligonucleotide is capable of targeting the derivatized RNA molecule to a subcellular location in a recipient cell.

22. The derivatized RNA molecule of claim 2, wherein the polypeptide comprises a therapeutic protein.

23. The derivatized RNA molecule of claim 22, wherein the therapeutic protein comprises Cystic Fibrosis Transmembrane Conductance Regulator (CFTR).


SEQUENCE LISTING (2 sequences)

SEQ ID NO: 1 - protein, 19 aa, synthetic construct
YRMQLLSCIALSLALVTNS

SEQ ID NO: 2 - protein, 11 aa, synthetic construct
FVFLVLLPLVS